(19) United States
(12) Patent Application Publication
(10) Pub. No.: US 2011/0147317 A1
(43) Pub. Date: Jun. 23, 2011

(54) Catalytic Compositions, Composition Production Methods, and Aqueous Solution Treatment Methods

(76) Inventors: Qi Li, Champaign, IL (US); Yin Wai Li, Mobile, AL (US); Pinggui Wu, Chicago, IL (US); Rongcai Xie, Ahqing city (CN); Jian ku Shang, Mahomet, IL (US)

(21) Appl. No.: 13/060,194

(22) PCT Filed: Aug. 24, 2009
(86) PCT No.: PCT/US09/54785

§ 371 Date: Feb. 22, 2011

Related U.S. Application Data
(60) Provisional application No. 61/091,314, filed on Aug. 22, 2008.

(51) Int. Cl. A61L 2/08; B01J 27/24; A01N 59/16; B01J 37/02; A01P 1/00; C02F 1/30; B01J 19/12

(52) U.S. Cl. 210/748.09; 502/200; 424/646

(57) ABSTRACT

Composition production methods are provided that can include providing PdO nanoparticles on a nitrogen-doped titanium oxide surface to form a catalytic mixture. Catalytic compositions and/or bactericides are provided that can include a substrate supporting Ti, O, N, and Pd. Water purification methods are provided that can include exposing an aqueous solution to a composition comprising at least a substrate supporting Ti, O, N, and Pd. Photocatalytic methods are provided that can include: providing a composition comprising one or both of Ti and Pd; exposing the composition to visible radiation to activate the composition; and in the substantial absence of the visible radiation, contacting the composition with an aqueous solution to purify the aqueous solution. Embodiments of the disclosure provide visible light photocatalysts that can demonstrate faster photocatalytic disinfection rates on Escherichia coli (E. coli) under visible light illumination as compared to nitrogen-doped titanium oxide (TiON), as well as catalytic activity after visible light illumination is substantially removed.

### CROSS REFERENCE TO RELATED APPLICATION

This application claims priority under 35 U.S.C. §119 to U.S. Provisional Patent Application Ser. No. 61/091,314 entitled “Photocatalysts and Photocatalytic Methods”, which was filed Aug. 22, 2008, the entirety of which is incorporated by reference herein.

### GOVERNMENT RIGHTS STATEMENT

This invention was made with Government support by the Center of Advanced Materials for the Purification of Water with Systems, National Science Foundation, under Agreement Number CTS-0120978; Grant Number DEFG02-91-ER45439 awarded by the U.S. Department of Energy. The Government has certain rights in the invention.

### TECHNICAL FIELD

The present disclosure relates to catalytic compositions, composition production methods, and aqueous solution treatment methods.

### BACKGROUND

Since the discovery of photoelectrochemical splitting of water on n-TiO₂ electrodes by Fujishima and Honda in 1972, semiconductor-based materials have been investigated extensively as photocatalysts for both solar energy conversion and environmental applications. Due to its high chemical stability, good photoactivity, relatively low cost and nontoxicity, TiO₂ has appeared as a candidate among various semiconductor-based photocatalysts, especially for industrial use. The photocatalytic capability of TiO₂ can require ultraviolet light (λ<400 nm) illumination due to its relatively wide band gap (˜3.2 eV for anatase TiO₂), which can limit its solar efficiency.

Anionic doping of n-TiO₂ photocatalyst can extend the optical absorbance of TiO₂ into the visible-light region. Anionic nonmetal dopants, such as nitrogen, carbon, sulfur, or fluorine, have been explored for visible-light photocatalysis so that a greater portion of the solar spectrum or just indoor light may be used to provide photocatalytic capability. Anion-doping can provide massive charge carrier recombination, which can limit the photoactivity of anion-doped TiO₂ under visible light illumination. Anion-doped TiO₂ photocatalysts can also lose their photocatalytic capability in the dark environment, where they may not produce electron and hole pairs.

It would be most desirable to design a visible-light photocatalyst system which can provide enhanced photocatalytic efficiency by minimizing charge carrier recombination. It would be even better if the improved photocatalyst can store some of its photocatalytic activity in “memory” so that once the photoexcitation is turned off, the catalyst still remains active for an extended period of time.

### SUMMARY

Catalytic compositions are provided that can include a substrate supporting Ti, O, N, and Pd.

Bactericides are provided that can include Ti, O, N, and Pd.

Composition production methods are provided that can include providing PdO nanoparticles on a nitrogen-doped titanium oxide surface to form a catalytic mixture.

Water purification methods are provided that can include exposing an aqueous solution to a composition comprising at least a substrate supporting Ti, O, N, and Pd.

Photocatalytic methods are provided that can include: providing a composition comprising one or both of Ti and Pd; exposing the composition to visible radiation to activate the composition; and in the substantial absence of the visible radiation, contacting the composition with an aqueous solution to purify the aqueous solution.

Embodiments of the disclosure provide visible light photocatalysts based on palladium oxide nanoparticles dispersed on nitrogen-doped n-TiO₂. These catalysts can be referred to as TiON/PdO. Implementations of the catalyst can demonstrate not only fast photocatalytic disinfection rates on Escherichia coli (E. coli) under visible light illumination as compared to nitrogen-doped titanium oxide (TiON), but, also a “memory” catalytic disinfection capability after visible light illumination is turned off for periods up to 8 hours.

### DRAWINGS

Embodiments of the disclosure are described below with reference to the following accompanying drawings.

FIG. 1 is a depiction of a composition production method according to an embodiment.

FIG. 2 depicts characteristics of embodiments of the compositions of the disclosure.

FIG. 3 depicts characteristics of embodiments of the compositions of the disclosure.

FIG. 4 depicts an aqueous solution purification method according to an embodiment.

FIG. 5 depicts an aqueous solution purification method according to an embodiment.

FIG. 6 depicts data achieved utilizing embodiments of the methods of the disclosure.

FIG. 7 depicts data achieved utilizing embodiments of the methods of the disclosure.

FIG. 8 depicts data achieved utilizing embodiments of the methods of the disclosure.

### DESCRIPTION

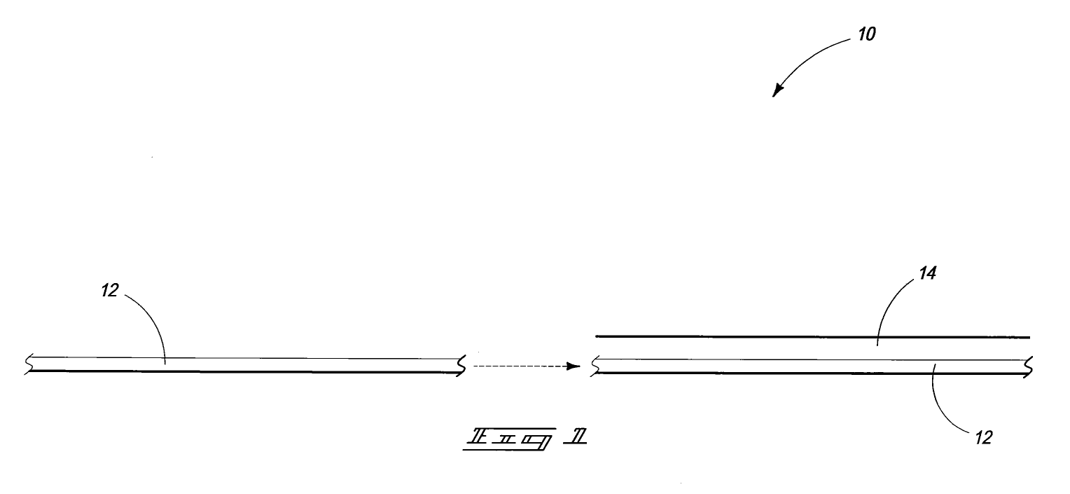

Embodiments of the catalytic compositions, composition production methods, and aqueous solution treatment methods with be described with reference to FIGS. 1-8. Referring first to FIG. 1, a composition production method 10 is shown. A substrate 12 is provided, and this substrate can include silicon such as glass. Substrate 12 can be a solid substrate. Substrate 12 can also be a mesh of silicon-comprising materials such as glass fibers, for example.

To substrate 12 can be provided mixture 14. Mixture 14 can include one or more Ti and transitional metal oxides such as Pd, Ag, Fe, and/or Cu. These metals can be in oxide form, for example, TiO₂, and PdO. Example compositions and methods for preparing same can be found in Published United States Patent applications: 20070190765 A1, filed Dec. 22, 2006 and entitled QUATERNARY OXIDES AND CATALYSTS CONTAINING QUATERNARY OXIDES; and 20070202334 A1, filed Dec. 22, 2006 and entitled NANOPARTICLES CONTAINING TITANIUM OXIDE, the entirety of both of which are incorporated by reference herein.

According to example implementations, production methods can include providing PdO nanoparticles on a nitrogen-doped titanium oxide surface to form a catalytic mixture. Mixture 14 can be prepared as a precursor solution. For example, preparation of palladium-modified nitrogen-doped titanium oxide (TiON/PdO) precursor solutions can be completed at room temperature in a sol-gel process. The process can include dissolving tetramethylammonium hydroxide (TMA, 25 wt % in methanol, Sigma-Aldrich, St. Louis, Mo., U.S.A.) in ethyl alcohol (EtOH, 100%, AAPER Alcohol and Chemical Co., Shelbyville, Ky., U.S.A.) at a mol ratio at 1:10. The solution can then be stirred magnetically for about 5 min, and titanium tetraisopropoxide (TTIP, 97%, Sigma-Aldrich, St. Louis, Mo., U.S.A.) can be added into the solution with the TMA:TTIP mol ratio at 1:5. A proper amount of palladium acetylacetonate (Pd(acac)₂, 99%, Sigma-Aldrich, St. Louis, Mo., U.S.A.) can be dissolved in dichloromethane (CH₂Cl₂, 99.6%, Sigma-Aldrich, St. Louis, Mo., U.S.A.), and then added into the TMA/TTIP/EtOH mixture to achieve desired Pd:Ti mol ratio at 0.5%.

After stirring for 5 min to get a homogeneous TiON/PdO precursor solution, substrate 12 such as activated carbon glass fiber can be soaked into it for 24 h. After being removed from the precursor solution and following washing in EtOH, the soaked template can be left in air and the hydrolysis of precursors initiated by exposure to the moisture in air. After further hydrolysis and drying, the composition can be calcined in air for 3 hours and a composition of TiON/PdO on glass fiber obtained. For comparison purpose, nitrogen-doped titanium oxide (TiON) photocatalytic fiber sample can be synthesized a similar method with the exception of the addition of palladium precursor.

In accordance with example implementations a catalytic composition comprising substrate 12 supporting mixture 14 can be prepared. Mixture 14 can include Ti, O, N, and Pd. The Ti can be in the form of TiO₂ and can be supplemented with N. As an example, the TiO₂ can be doped with N to form n-TiO₂ particles. The Pd of mixture 14 can be dispersed on the n-TiO₂ particles, for example. The Pd is in the form of PdO. Supported composition 14 can be photocatalytic and can remain catalytically active in the absence of visible radiation. Supported composition 14 can be a bactericide, for example.

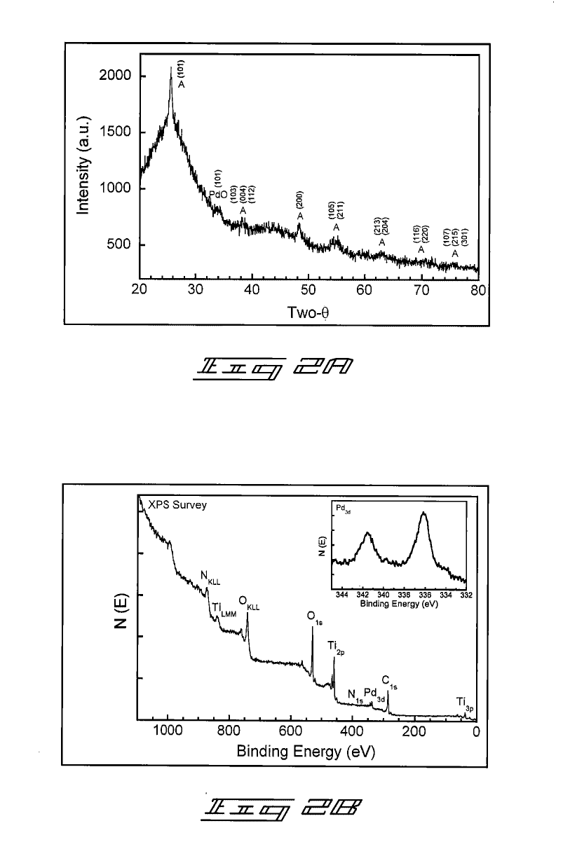

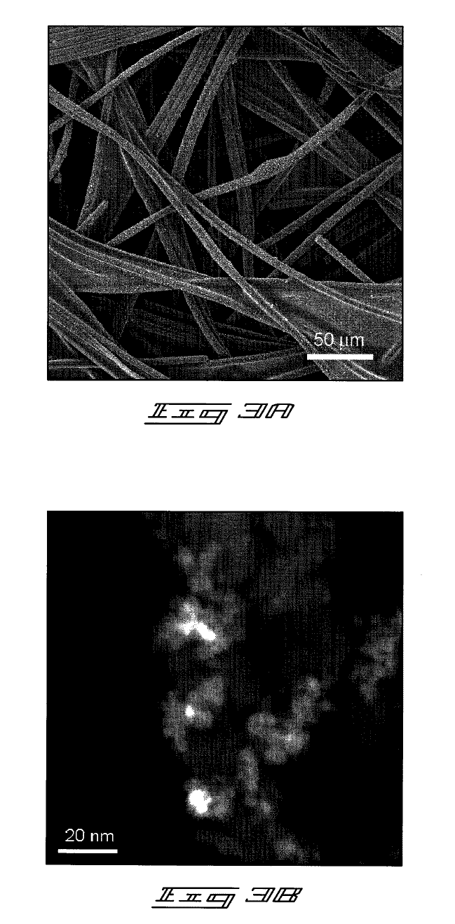

Referring to FIGS. 2 and 3, data and crystal structures of supported composition 14 are depicted. Multiplex XPS spectra of N 1s, O 1s, Ti 2p, and Pd 3d can be recorded using band-pass energy of 35.75 eV corresponding to an energy resolution of 1.2 eV. Atomic concentrations of these elements can be obtained by comparing the peak areas of their spectra. The morphology of the fiber can be examined by scanning electron microscopy (SEM) with a Hitachi S-4700 Scanning Electron Microscope (Hitachi Ltd., Tokyo, Japan). Prior to imaging, the sample can be sputtered with gold for 15 seconds (Emitech K575 Sputter Coater, Emitech Ltd., Ashford Kent, UK). Scanning transmission electron microscopy (STEM) can also used to provide detailed information of the fiber surface on a JEOL 2010F transmission electron microscope (JEOL Ltd., Tokyo, Japan) operated at 200 kV. The UV-vis spectra of TiON/PdO and TiON powders can be measured on a Cary 500 UV/Vis/NIR spectrophotometer (Varian, Inc., Palo Alto, Calif., U.S.A.).

Referring first to FIG. 2(a) an X-ray diffraction (XRD) pattern of the supported composition is shown, while 2 (b) depicts the representation of an XPS survey spectrum of the supported composition 14. Reflection peaks can demonstrate that TiON/PdO fiber has a dominant anatase structure without rutile phase, which is similar to TiON fiber samples. A very weak peak belonging to PdO (101) can be observed in the XRD pattern of the TiON/PdO sample, which can suggest that Pd additive exists as PdO at a very small quantity and is not incorporated into the dominant anatase structure.

FIG. 2b depicts the representative XPS survey spectrum of TiON/PdO fibers, which can demonstrate the existence of N, O, Pd and Ti. Due to the widespread presence of carbon in the environment, C 1s peak is also in XPS survey spectrum. The relative elemental composition ratio can be determined by multiplex high-resolution scans over N 1s, O 1s, Pd 3d, and Ti 2p spectral regions. An average surface N/Ti atomic ratio of ˜0.10 can be found, and the surface Pd/Ti atomic ratio can be at ˜0.03. The inset plot demonstrates the magnified survey spectra over Pd 3d spectral region of TiON/PdO fibers and depicts the existence of Pd additive as PdO in TiON/PdO powder because the binding energy of Pd 3d_{5/2} is ˜336.20 eV, which is in agreement with the XRD observation.

FIG. 3(a) represents an SEM image, and 3(b) an STEM image of supported composition 14. Supported compositions 14 can be analyzed by X-ray diffraction (XRD) on a Rigaku D-Max X-ray powder diffractometer (Rigaku Corporation, Tokyo, Japan) with Ni-filtered Cu (0.15418 nm) radiation at 45 kV and 20 mA. XPS measurements can be made on Physical Electronics PHI 5400 X-ray Photoelectron Spectrometer (Perkin-Elmer Corporation, Eden Prairie, Minn., U.S.A.) with an Mg K anode (1253.6 eV photon energy, 15 kV, 300 W) at a take-off angle of 45°. The SEM image of the TiON/PdO photocatalytic fibers (FIG. 3a) can demonstrate that the fiber network of the ACGF template matrix can be maintained and a thin layer of TiON/PdO photocatalyst immobilized on the glass fiber template surface. The fiber network can increase the contact efficiency for bacterial removal. STEM image of TiON/PdO fibers is depicted in FIG. 3b, which can demonstrate that two distinct kinds of particles co-exist on the glass fiber surface. Grey ones with larger particle size (˜5 to 10 nm in diameter) can be recognized as TiON, while bright ones with smaller particle size (˜1 to 2 nm in diameter) can be recognized as PdO. This observation can be relied upon to support the belief from XRD analysis that Pd additive exists as PdO not incorporated into the dominant anatase structure, and is in accordance with the observation on TiON/PdO thin film fabricated by ion-beam-assisted-deposition (IBAD). The Pd/Ti atomic ratio determined by XPS (˜0.03) can be higher than that in the sol-gel precursor solution. As a surface characterization technique, XPS can only determine the surface composition ratio within a very shallow depth. Due to the enrichment of palladium additives on the fiber surface, a higher Pd/Ti atomic ratio by XPS than that in the precursor ratio solution can be expected.

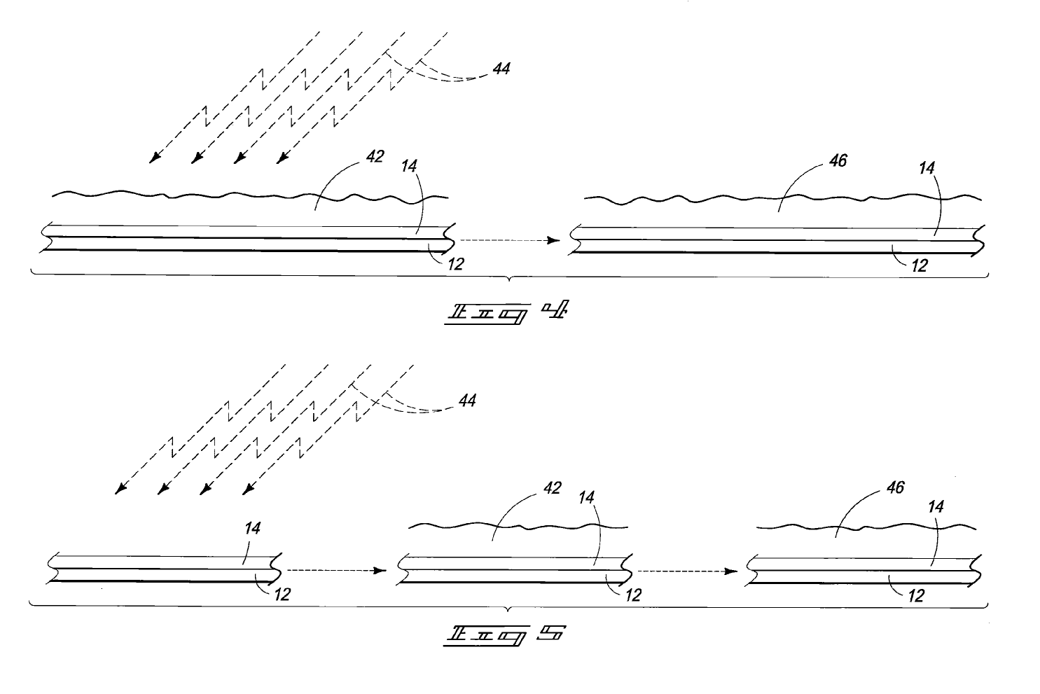

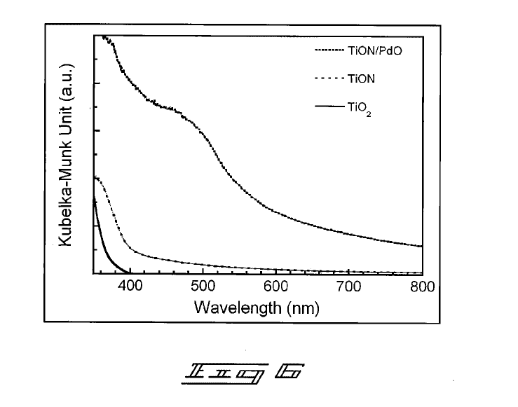

Referring next to FIGS. 4 and 5, water purification and/or photocatalytic methods are depicted. Referring first to FIG. 4, the composition comprising substrate 12 supporting mixture 14 can be exposed to aqueous solution 42. Aqueous solution 42 can be a solution in need of treatment such as contaminated and/or infected water for example. Solution 42 can include bacteria and/or other contaminants whose removal is desired for environmental, health, and/or sanitary purposes. As an example, solution 42 can be water that is in need of purification. The purification can be a disinfection and/or bactericidal.

The mixture 14 of the composition can be exposed to visible radiation 44. This can activate the composition, for example. As shown in FIG. 4, mixture 14 can be exposed to radiation 44 while being exposed to solution 42. Catalytically activated mixture 14 can provide substantially purer aqueous solution 46, solution 46 being free from at least some of the undesirable contaminants of solution 44, for example.

Referring to FIG. 5, mixture 14 can be exposed to radiation 44 in the absence of a solution to be purified and then in the substantial absence of radiation 44, solution 42 can be exposed to mixture 14.

Mixture 14 can be catalytically activated in the absence of the solution and then exposed to solution 42 to purify solution 42 and form solution 46.

As part of either of the methods depicted in FIGS. 4 and/or 5, the providing of radiation 44 can be substantially ceased either before, during or after exposing mixture 14 to solution 42. Upon removal of the radiation, mixture 14 can remain catalytically active to purify the aqueous solution.

As an example, the method can include providing a composition comprising one or both of Ti and Pd, such as mixture 14. The composition can be exposed to visible radiation to activate the composition, and, in the substantial absence of the visible radiation, the composition can be contacted with an aqueous solution to purify the aqueous solution.

Without limiting the scope of the disclosure, it may be that these antimicrobial properties of TiON/PdO may be derived from the optoelectronic coupling between the PdO nanoparticles and the TiON semiconductor, which can promote the charge carrier separation in TiON and results in the chemical reduction of PdO to Pd⁰. While the separation of the charge carriers can enhance the visible light photocatalytic killing of E. coli, a “memory” antimicrobial effect may result from the catalytic effect of Pd⁰. The antimicrobial effects of TiON/PdO photocatalyst under visible light illumination and their post-illumination activity provide for new implementations, such as continuous solar-powered disinfection during daytime and at night, for a broad range of environmental applications.

The optical property of TiON/PdO can be compared with both TiON and TiO₂. The optical absorbance of TiON/PdO can be determined from the diffuse reflectance measurements of sol-gel TiON/PdO nanoparticle powders, which can be synthesized with the same sol-gel process as the TiON/PdO fibers without utilizing the fiber soaking procedure. These TiON/PdO nanoparticle powders have similar crystal structure and composition with their counterparts in the fiber form and can be used in the optical measurement here for the experimental convenience. The optical absorbance can be approximated from the reflectance data by the Kubelka-Munk function, as given by Eq. (1):

$$F ( R ) = \frac{{( 1 - R )}^{2}}{2 R} \qquad ( 1 )$$

where R is the diffuse reflectance.

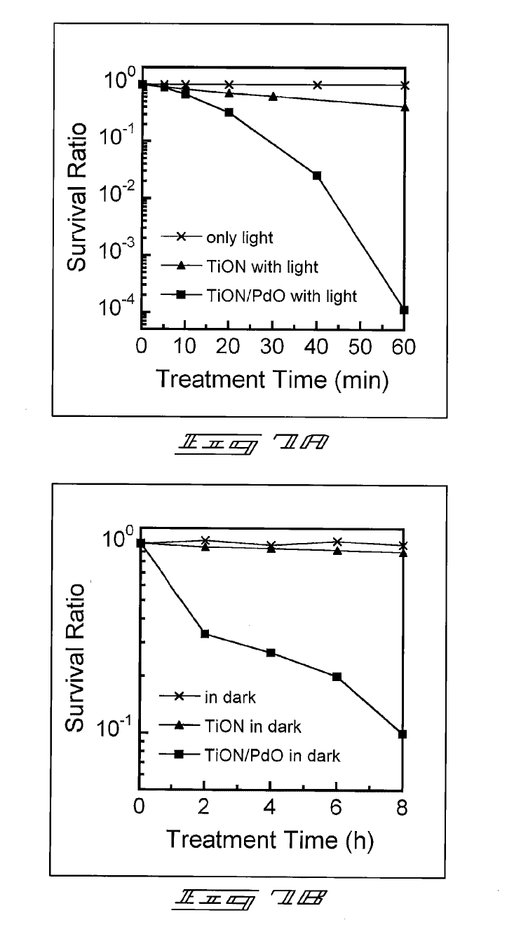

FIG. 6 depicts the light absorbance (in term of Kubelka-Munk equivalent absorbance units) of sol-gel TiON/PdO nanoparticle powders, compared with the light absorbance of sol-gel TiON nanoparticle powders and Degussa P25 powders. TiON nanoparticle powders can be synthesized under the same condition as TiON/PdO samples except for the addition of palladium precursor, and Degussa P25 powder is a commercial TiO₂ powder that is widely studied in photocatalysis. Degussa P25 demonstrates characteristic spectrum with the fundamental absorbance stopping edge at ˜400 nm, which can suggest that its photocatalytic capability is limited mainly in the UV light region. TiON powders demonstrate a shift into the visible light range (>400 nm), which can be attributed to the nitrogen dopant effect. TiON/PdO powders show much higher visible light absorption than TiON powders in the whole visible light region, which can suggest that Pd additive promotes visible light absorption in TiON. The enhanced visible light absorption is attributed to the surface plasma resonance effect from metallic Pd⁰ nanoparticles under visible light illumination.

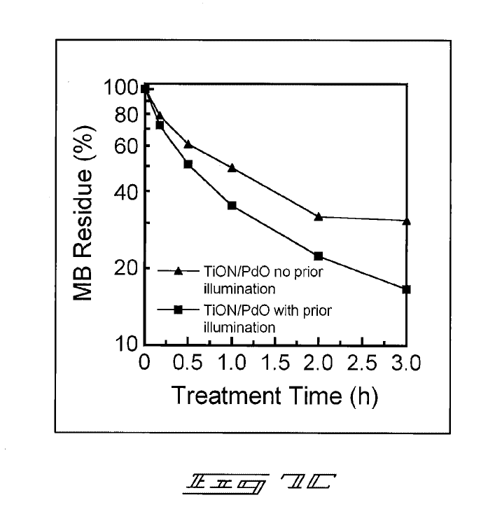

The photocatalytic activity of TiON/PdO can be measured by testing on wild type E. coli AN 387. For this purpose, E. coli cells can be suspended in a buffer solution with TiON/PdO fiber samples and exposed to visible light (λ>400 nm) for varying time intervals. The same procedure can be followed for the TiON fiber samples. The buffer solution itself may keep E. coli alive up to 2 weeks. The survival ratio of E. coli can be determined by the ratio of Nₜ/N₀, where N₀ and Nₜ are the numbers of colony-forming units at the initial and each following time interval, respectively. FIG. 7A depicts the E. coli survival ratio under visible light illumination with different treatments. Without photocatalysts, no bactericidal effect can be observed under visible light illumination. A bactericidal effect can be observed on TiON photocatalytic fiber under visible light illumination, which is consistent with its demonstrated visible light absorption capability. After 1 h visible light illumination, ˜60% E. coli cells can be inactivated under TiON treatment. Compared with TiON, TiON/PdO photocatalytic fiber may show a faster bactericidal effect when compared to E. coli under visible light illumination. After only one hour visible light illumination, the E. coli survival ratio can drop to ˜10⁻⁴ under TiON/PdO fiber treatment, which is over 3 orders of magnitude lower than the survival ratio reached with TiON. Thus, the visible-light-induced photocatalytic efficiency of TiON can improved by addition of PdO nanoparticles.

The disinfection properties of TiON/PdO can be further examined after light exposure. Photocatalytic fiber samples can first be illuminated by the same lamp for ˜10 h to simulate the day-time visible light illumination condition. The lamp can then be switched off, and the disinfection of E. coli conducted in the same experimental setup as the photocatalytic disinfection experiment described above, only without the light illumination. FIG. 7B depicts the E. coli survival ratio in dark under the three different treatments. Without photocatalysts, no bactericidal effect can be observed in the dark. TiON photocatalytic fiber can demonstrate no significant bactericidal effect in the dark either, because of the lack of light absorption and subsequent electron-hole pair production, for example. TiON/PdO photocatalytic fibers can display a striking bactericidal capability in the dark environment. The bactericidal effect can persist even after several hours in the dark environment. After 8 h, about 90% E. coli cells can be inactivated of the initially fresh E. coli cell suspension (ca. 10⁷ cfu/ml).

To clarify the role of PdO nanoparticles in the enhancement of photocatalytic disinfection efficiency under visible light illumination and the “memory” catalytic disinfection capability after the light is off, two comparison experiments can be conducted. TiO₂/PdO can be prepared with the same procedure as TiON/PdO, but without the nitrogen doping. TiO₂/PdO may show little bactericidal effect under the similar visible light illumination or after light exposure. The TiON/PdO fiber can be kept in the dark for more than 24 h after the light exposure, before using them to interact with E. coli without light illumination and no significant bactericidal effect can be observed. TiON/PdO by itself is not toxic to E. coli. Pd dopants themselves, including Pd species possibly leached into the E. coli cell suspension, could not contribute much to the disinfection of E. coli. Otherwise, high disinfection efficiency would be observed in TiO₂/PdO or TiON/PdO with/without light illumination. The enhancement of photocatalytic disinfection efficiency under visible light illumination may be related to the interaction between TiON and PdO, and the bactericidal effect observed in the dark is rather related to the prior illumination of TiON/PdO by visible light before the light is turned off, hence the “memory” effect.

The “memory” effect can be further examined by investigating the interactions between TiON/PdO fiber samples with a commonly used model pollutant, methylene blue (MB), without the light illumination. Two TiON/PdO samples can first be kept in dark for 24 h. One sample can then be illuminated by the same lamp for ˜10 h to simulate the day-time visible light illumination condition, while the other sample can still be kept in dark. After the lamp may be switched off, the interaction between TiON/PdO fiber and MB solution on both samples can be observed without the light illumination. After varying time intervals, the light absorption of the clear solution can be measured and the remaining percentage of MB in the solution can be calculated by the ratio between the light absorptions of TiON/PdO treated and untreated MB solutions. FIG. 7C provides a summary of the residual percentage of MB as a function of treatment time. MB concentration in the solution decreases under the treatments of both samples, while the one treated by TiON/PdO fiber sample with prior visible light illumination shows a lower residual MB concentration at each time interval. Unlike the living E. coli cells, which are very active and could not be easily adsorbed, organic pollutants in the aqueous solution could be easily adsorbed by the TiON/PdO fiber network. This explains the decrease of MB concentration in the solution treated by TiON/PdO fiber sample without prior visible light illumination. With the increase of the interaction time, the adsorption process gradually reaches the equilibrium after around 2 h, and no further decrease of MB residual concentration can be observed. The MB residual concentration can continue to decrease on the solution treated by TiON/PdO fiber sample with prior visible light illumination. After 3 h treatment, the MB residual concentration can decrease to around 16% of the initial value. This observation may suggest that the further decrease of MB residual concentration is due to the degradation other than the adsorption effect of TiON/PdO fiber, which is due to the “memory” effect related to the prior illumination of TiON/PdO by visible light before the light is turned off.

In the TiON/PdO photocatalytic material system, electron-hole pairs can be produced under visible light illumination. The changes in PdO nanoparticles following light exposure can be examined by an in-situ XPS analysis, which showed the reduction of PdO to metallic Pd⁰.

When electrons move from TiON to PdO nanoparticles, the PdO semiconductor nanoparticles can be reduced on the surface to metallic Pd⁰ nanoparticles so that these electrons are locally trapped at these nanoparticles. The trapping of charge carriers can decrease the e⁻/h⁺ pair recombination rate and subsequently increase the lifetime of charge carriers, which can be beneficial to improve the photoactivity, as observed in FIG. 7A. When the visible light is shut off, the metallic Pd⁰ nanoparticles can discharge the electrons to the TiON matrix, which may react with oxygen/water to produce radicals by the following reaction:

O₂+e⁻→O₂⁻ (2)

2O₂⁻+2H⁺→2.OH+O₂ (3)

Both O₂⁻ and .OH are highly reactive radicals, which can provide TiON/PdO with the post-exposure catalytic disinfection capability on E. coli bacteria and degradation effect on MB.

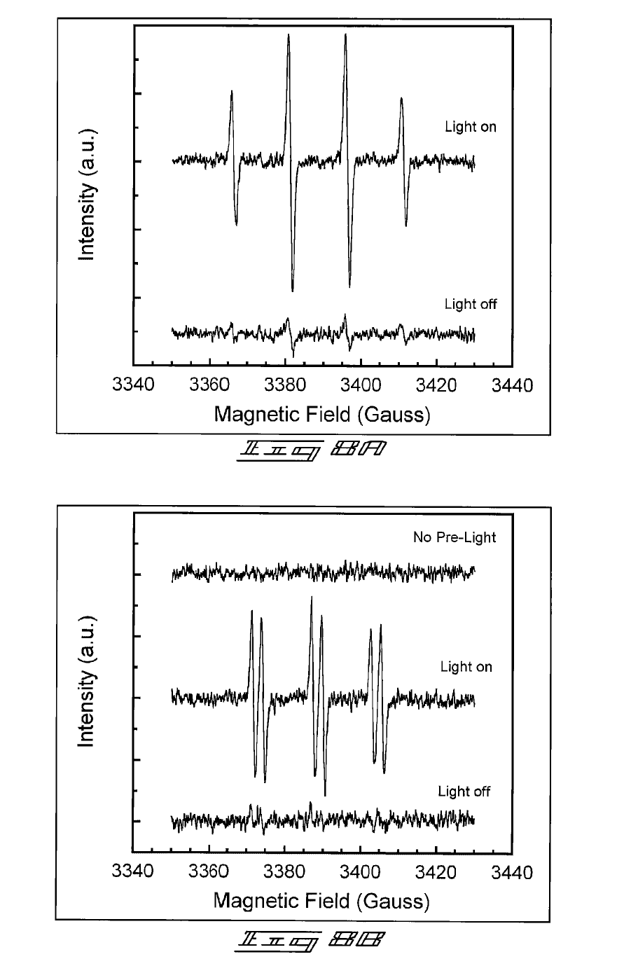

To verify the formation of reactive radicals by TiON/PdO, spin trapping EPR measurements can be conducted on TiON/PdO sol-gel powders both under visible light illumination and in the dark with two different spin trapping chemicals, including DMPO and POBN. Before the measurement, a Fenton reaction can be first conducted in comparison and as proof of the correct instrumentation. FIG. 8A demonstrates the EPR spectra of TiON/PdO samples under the interaction with DMPO at different experimental conditions. Under the visible light illumination, the four-peak DMPO-.OH adduct signals can be observed, which may demonstrate the production of .OH radicals by TiON/PdO under visible light illumination. The same measurement in the dark on a sample with pre-exposure can be conducted. While the four-peak DMPO-.OH adduct signals may be weaker than that under visible light illumination, they may still be distinguished easily.

Compared with DMPO which may interact with various radicals, POBN can specifically interact with .OH radicals. Thus, the spin trapping EPR measurement can be furthered with POBN. FIG. 8B demonstrates the EPR spectra of TiON/PdO samples under the interaction with POBN at different experimental conditions. Without pre-exposure on TiON/PdO sample and the measurement conducted in dark, no POBN-.OH adduct signals can be observed, which is consistent with the observation of little bactericidal effect of TiON/PdO fiber kept in dark for more than 24 h before being used to interact with E. coli without light illumination. Under the visible light illumination, the strong characteristic triple-peak POBN-.OH adduct signals can be observed, which demonstrates the production of .OH radicals by TiON/PdO under visible light illumination. The measurement conducted in dark on TiON/PdO sample with pre-exposure also demonstrates triple-peak POBN-.OH adduct signals, which is in accordance with the EPR measurement result with DMPO. Spin trapping EPR measurements suggest that the “memory” effect did produce .OH radicals when the visible light is shut off. It should be noted that what determines the duration of the “memory” effect is the rate of charge release from Pd⁰ particles and this duration should not be confused with the lifetime of the radicals.

Although the post-illumination catalytic disinfection efficiency is not as high as the photocatalytic disinfection efficiency under visible light illumination, the inactivation rate from the “memory” effect is still comparable to those of TiON powder under visible light illumination or TiO₂ under UV irradiation, which are roughly 50% reduction in E. Coli concentration after 4 h of light or UV activation. After 8 h of stay in the dark, the inactivation rate of TiON/PdO may not decay rapidly to a plateau in FIG. 7B, suggesting that disinfection may persist for additional hours. With such a persistent dark disinfection capability, TiON/PdO photocatalyst can offer a unique catalytic material for solar-based disinfection system where during the day time (or when visible light illumination is present), the photocatalytic disinfection prevails and at night (or when visible light is absent), the catalytic “memory” effect takes over.

TiON/PdO photocatalytic fibers can be synthesized by adding PdO nanoparticles on nitrogen-doped titanium oxide surface. The TiON/PdO fibers may demonstrate high disinfection efficiency on E. coli bacteria under visible light illumination, and most strikingly a post-illumination catalytic disinfection capability after the visible light is shut off. Such a unique combination of strong photocatalytic disinfection with persistent post-illumination catalytic disinfection properties come from the electron transfer in and out of PdO nanoparticles during and after visible light illumination of nitrogen-doped titanium oxide. It is believed that such charge transfers may also occur for other transition metal oxide nanoparticles dispersed on anion-doped n-TiO₂ photocatalyst systems. The resulting combination of photocatalytic and post-illumination catalytic properties makes these materials suited for solar-based systems in a broad range of environmental applications.

Wild type Escherichia coli AN 387 (ATCC 15597, the American Type Culture Collection, Manassas, Va., U.S.A.) can be used for photocatalytic inactivation experiment. After overnight culture, the cells can be diluted to a cell suspension (ca. 10⁷ cfu/ml) in buffer solution (0.05 M KH₂PO₄ and 0.05 M K₂HPO₄, pH 7.0) prior to the use for photocatalytic inactivation. All solid or liquid materials can be autoclaved for 30 min at 121° C. before use. For E. coli disinfection under visible light illumination, a metal halogen desk lamp can be used, which has a glass filter to provide zero light intensity below 400 nm. The light intensity striking the cell suspensions can be at ca. 1.0 mW/cm², as measured by a Multi-Sense MS-100 optical Radiometer (UVP, Inc., Upland, Calif., U.S.A.). For E. coli disinfection under dark environment, photocatalytic fiber samples can first be illuminated by the same lamp for ˜10 h to simulate the day-time illumination, then the lamp can be shut off and fiber samples used to conduct disinfection experiment in dark over fresh E. coli cell suspension (ca. 10⁷ cfu/ml). At the starting time, aliquot of 3-mL E. coli cell suspension may be pipetted onto a sterile 60×15 mm petri dish with photocatalytic fiber sample placed in the bottom. A fixed concentration of ˜1 mg photocatalyst/mL E. coli solution can be used in this study. At regular time intervals, 20 μL of aliquots of the TiON/PdO-treated cell suspensions may be withdrawn in sequence. After appropriate dilutions in buffer solution, aliquots of 20 μL together with 2.5 mL top agar may be spread onto an agar medium plate and incubated at 37° C. for 18 h. The number of viable cells in terms of colony-forming units may be counted. Analyses may be performed in duplicate with control runs carried out each time under the same experiment conditions, but without any photocatalytic materials.

Methylene blue may be used for static interaction experiment with TiON/PdO fiber samples in the dark. Two TiON/PdO samples may first be kept in dark for 24 h. One sample can then be illuminated by the same lamp for ˜10 h to simulate the day-time visible light illumination condition, while the other sample can still be kept in dark. The fiber sample may be placed at the bottom of 45×10 mm petri dishes, and 2 ppm MB solution may be added into the petri dish at a fixed concentration of 0.5 mg photocatalyst/mL solution. The covered petri dish can be kept in dark during the treatment process. The treatment time can vary from 10 min to 3 h. After the treatment, the light absorption of the clear solution can be measured by a Cary 500 UV/Vis/NIR spectrophotometer. The remaining percentage of MB in the solution can be calculated by the ratio between the light absorptions of TiON/PdO treated and -untreated MB solutions.

Spin trapping electron paramagnetic resonance (EPR) measurements can be conducted to verify the formation of reactive radicals by TiON/PdO photocatalytic powders both under visible light illumination and in the dark. Two spin trapping chemicals can be used in this study, including 5,5-Dimethyl-1-pyrroline N-oxide (DMPO, 97%, Sigma-Aldrich, St. Louis, Mo., U.S.A.) and α-(4-Pyridyl N-oxide)-N-tert-butylnitrone (POBN, 99%, Sigma-Aldrich, St. Louis, Mo., U.S.A.). DMPO may first be decolorized by active carbon and then filtered with millipore filter to get rid of impurities before the use. Before the measurements with TiON/PdO, a Fenton reaction can first be conducted as comparison and as proof of the correct instrumentation. TiON/PdO powders can be put into petri dishes and dispersed in double deionized water before adding electron trapping chemicals. DMPO can be prepared as a 100 mM stock solution and the final concentration of DMPO within the TiON/PdO dispersion can be 50 mM. 100 mM POBN and 95% ethanol stock solution can be added into the TiON/PdO dispersion to reach a final concentration of POBN at 10 mM and ethanol at 170 mM. EPR spectra can be collected on a Eline Century Series EPR Spectrometer (Varian E-109-12, Varian, Inc., Palo Alto, Calif., U.S.A.) working in the X-band mode at 9.51 GHz, center field 3390 G, and 10.00 dB power.

1. A catalytic composition comprising a silicon-comprising substrate supporting Ti, O, N, and Pd.

2. The composition of claim 1 wherein the substrate comprises a mesh material.

3. (canceled)

4. The composition of claim 1 wherein the substrate comprises glass fibers.

5. The composition of claim 1 wherein the Ti is in the form of TiO₂.

6. (canceled)

7. The composition of claim 5 wherein the TiO₂ is doped with N to form n-TiO₂ particles.

8. The composition of claim 7 wherein the Pd is dispersed on the n-TiO₂ particles.

9. (canceled)

10. (canceled)

11. (canceled)

12. A bactericide comprising Ti, O, N, and Pd.

13. (canceled)

14. The bactericide of claim 12 wherein the bactericide is photocatalytic.

15. The bactericide of claim 12 wherein the bactericide is catalytically active in the absence of visible radiation.

16. (canceled)

17. (canceled)

18. A composition production method comprising providing PdO nanoparticles on a nitrogen-doped titanium oxide surface to form a catalytic mixture.

19. The method of claim 18 further comprising depositing the catalytic mixture on a solid substrate.

20. (canceled)

21. The method of claim 19 wherein the solid substrate comprises a mesh of silicon-comprising materials.

22. The method of claim 18 wherein the mole ratio of Pd:Ti is 0.5%.

23. A water purification method comprising exposing an aqueous solution to a composition comprising at least a substrate supporting Ti, O, N, and Pd.

24. (canceled)

25. (canceled)

26. The method of claim 23 further comprising exposing the composition to visible radiation to activate the composition.

27. The method of claim 26 wherein the solution is exposed to the composition while the composition is exposed to the visible radiation.

28. The method of claim 26 further comprising substantially ceasing the exposure of the composition to the visible radiation.

29. (canceled)

30. A photocatalytic method comprising:
providing a composition comprising one or both of Ti and Pd;
exposing the composition to visible radiation to activate the composition; and
in the substantial absence of the visible radiation, contacting the composition with an aqueous solution to purify the aqueous solution.

31. (canceled)

32. (canceled)

33. (canceled)

34. (canceled)

35. The method of claim 30 further comprising disinfecting the aqueous solution.

36. The method of claim 30 further comprising exposing the composition to visible radiation while contacting the composition with the aqueous solution.
